(19) United States
(12) Patent Application Publication
(10) Pub. No.: US 2010/0069544 A1
(43) Pub. Date: Mar. 18, 2010

(54) HYDROGEL CONTACT LENS COMPRISING A POLYMER COMPRISING A CARBOXY BETAINE ESTER MONOMER

(75) Inventors: Bernardus Franciscus Maria Wanders, Angerlo (NL); Henk Haitjema, Peize (NL)

Correspondence Address: FOLEY AND LARDNER LLP;SUITE 500, 3000 K STREET NW, WASHINGTON, DC, 20007, US

(73) Assignee: Procornea Holding B.V.

(21) Appl. No.: 12/516,274

(22) PCT Filed: Nov. 29, 2007
(86) PCT No.: PCT/NL07/50603

§ 371 Date: Oct. 29, 2009

Related U.S. Application Data
(60) Provisional application No. 60/867,722, filed on Nov. 29, 2006.

(30) Foreign Application Priority Data
Nov. 29, 2006 (EP) 06124997.5

(51) Int. Cl. C08K 5/20; C08F 220/54; C07C 229/02

(52) U.S. Cl. 524/217; 526/304; 560/169

(57) ABSTRACT

Hydrogel contact lens comprising a polymer composition obtainable by polymerising a monomer mixture comprising (a) a betaine ester and (b) an alkyl (meth) acrylate. The hydrogel contact lens has a high affinity for water, high water content and a high water retaining capability and provides a contact lens wearer with a high wearing comfort.

### FIELD OF THE INVENTION

The present invention relates to a hydrogel contact lens with a high affinity for water, high water content and having a high water retaining capability. A contact lens with these properties provides the contact lens wearer with a high wearing comfort.

### BACKGROUND OF THE INVENTION

The inner part of the natural cornea of the eye consists of a layer of endothelial cells that is provided with oxygen from the outer air by diffusion through the outer side of the cornea. In order to survive, the cornea thus has to permit a high oxygen flux to keep its own endothelial cells alive, which would be attainable when the cornea would be permanently exposed to outer air. However, then the cornea would dry out.

To avoid this, the cornea is wetted repeatedly by eyelid movements which spread tear fluid over the cornea, keeping it moist and thus permitting oxygen. This tear fluid contains proteins, lipoproteins, lipids and such, which would settle continuously on the cornea when the water in the tear film evaporates in the outer air when the cornea would be continuously exposed to outer air.

Nature has developed the cornea to solve this dilemma, which possesses contradicting properties like a high oxygen flux, a good wettability, high water content, a high water retaining capability and a high resistance to tear fluid deposits at the same time, yielding a clear, moist cornea. Thus far not many contact lens materials have been developed which sufficiently mimic these properties of the natural cornea in a contact lens.

U.S. Pat. No. 2,777,872 and U.S. Pat. No. 2,834,758 of E.I. DuPont de Nemours, incorporated by reference, disclose hydrophilic copolymers of N-(meth)acrylamidoalkyl carboxy betaine ester monomers, said hydrophilic copolymers being suitable as water-permeable colloids and useful for the manufacture of photographic emulsions and gelatine substitutes. The application of these polymers in contact lenses is not disclosed.

U.S. Pat. No. 2,935,493 and U.S. Pat. No. 2,958,682 of American Cyanamid Co., incorporated by reference, disclose copolymers of acryl amide or acrylonitril and N-acrylamidopropyl-N-carboxymethyl ammonium betaine. These copolymers can be used in plastics, coatings, adhesives, laminating and moulding. The application of these polymers in contact lenses is not disclosed.

U.S. Pat. No. 3,671,502 of Kendall Co., incorporated by reference, discloses hydrophilic copolymers of carboxy betaines or sulfobetaines and hydroxyalkyl(meth)acrylates which are useful as hydrophilic bonding agents for glass, leather, plastic, steel, hides and wood. The carboxy betaines include N-(meth)acylamidoalkyl-N-carboxyalkyl ammonium betaines. The application of these polymers in contact lenses is not disclosed.

U.S. Pat. No. 6,486,333 to Kao Corporation, incorporated by reference, discloses N-alkenylamidooxyalkylene carboxy betaine esters fur use as alcohol releasing substance in detergent compositions. The application of these polymers in contact lenses is not disclosed.

US 2003/0108513 of Goldschmidt AG, incorporated by reference, discloses N-(meth)acryloylalkyl carboxy betaine esters for hair conditioning formulations. The application of these polymers in contact lenses is not disclosed.

U.S. Pat. No. 5,311,225 of Johnson & Johnson Vision Products Inc. discloses hydrogel contact lenses manufactured from a polymer comprising the reaction product of hydroxyalkyl(meth)acrylates and a hydrophilic monomer, preferably a sulfobetaine, in particular N-(3-sulfopropyl)-N-methacryloyloxyethyl-N,N-dimethyl ammonium betaine (SPE).

U.S. Pat. No. 5,936,703 of NOF Corporation, incorporated by reference, discloses a contact lens having an enhanced hydrophilicity, wherein the contact lens is treated with a solution comprising an alkoxy silanyl carboxy betaine or sulfo betaine.

U.S. Pat. No. 6,733,123 of Wohlk Contact-Linsen GmbH, incorporated by reference, discloses hydrogel contact lenses manufactured from (meth)acryloyl amino acids, sulfo betaines such as SPE and a hydrophylic monomer selected from the group consisting of hydroxyethyl methacrylate (HEMA), hydroxypropyl methacrylate (HPMA), vinylpyrrolidone and an acrylamide derivative, preferably a dimethyl acrylamide derivative.

US 2005/0191335 of CooperVision Inc. and Ocular Sciences Inc., incorporated by reference, discloses hydrophilic lenses manufactured of a water-swellable, cross-linked polymeric material and a water swellable polymeric material, wherein the latter is physically immobilised by the cross-linked polymeric material. The hydrophilic lenses exhibit an increased water content, increased water retention and reduced water evaporation. The water swellable, cross-linked polymeric material may comprise betaines, e.g. sulfo betaines, carboxy betaines and phosphor betaines. Examples of the carboxy betaines are N-methacryloyloxyethyl-N,2-ethylcarboxy betaine, N-(3-carboxypropyl)-N-methylamino-ethylmethacrylate, N-(3-carboxypropyl)-1-methylaminomethacryloyloxyethyldimethylammonium betaine (CPE) and N-(3-carboxypropyl)aminoethylmethacrylate. None of these betaines are betaine esters. These betaines may be cross-linked with di(meth)acrylates such as ethylene glycol dimethacrylate (EDGMA).

JP 6067122 of Nippon Oils & Fats Co., Ltd., published 3 Nov. 1992, discloses a soft contact lens having a high moisture content and a high oxygen permeability, said soft contact lens being manufactured by polymerising a carboxy betaine and an ethylenically unsaturated monomer.

U.S. Pat. No. 6,590,051 of Ondeo Nako Company, published 8 Jul. 2003, discloses high molecular weight zwitter-ionic polymers comprising about 90 to about 97 mol % of a non-ionic monomer and about 3 to about 11 mol % of a zwitter-ionic monomer. The zwitter-ionic monomer may be a carboxy betaine which may be prepared by different methods as is shown in Scheme 2 of U.S. Pat. No. 6,590,051.

EP A 1.760.098 of Osaka Organic Chemical Ind., Ltd, published 7 Mar. 2007, discloses a biocompatible material comprising a polymer comprising a amino-acid type betaine monomer which is preferably N-methacryloyloxyethyl-N,N-dimethyl ammonium-α-N-methyl carboxy betaine. Such betaines may me prepared by different methods as is disclosed in paragraph [0019] of EP A 1.760.098. The biocompatible material may be used in food, as food additive, as a medicament, a quasi-drug, in a medical device, in cosmetics, in a toiletry article and the like. The medical device may be a contact lens.

However, all these hydrophilic lenses suffer from disadvantages and there is a continuous need in the art for hydrophilic contact lenses that have a superior oxygen flux, an excellent wettability, high water content, a high water retaining capability as well as a high resistance to tear fluid deposits.

Another feature of betaines is that they possess antimicrobial characteristics, see for instance U.S. Pat. No. 6,384,266 B1 to Farone et al., Applied Power Systems Inc, and EP 1000544 A1 to Stelter et al., Cognis Deutschland GmbH, both incorporated by reference herein. As the current carboxy betaine esters also contain the carboxy betaine structure that is thought to be responsible for the antimicrobial characteristics, the carboxy betaine esters may also possess similar antimicrobial characteristics.

### SUMMARY OF THE INVENTION

The present invention relates to a hydrogel contact lens comprising a polymer composition obtainable by polymerising a monomer mixture comprising (a) a betaine ester of the formula (I):

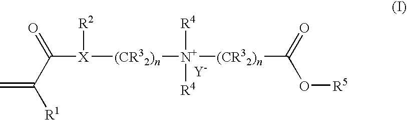

wherein:
R¹ is hydrogen or methyl;
X is O or N;
when X═N, R² is hydrogen or a linear, branched or cyclic C₁-C₁₂ alkyl group;
when X═O, R² is absent;
R³ is hydrogen or a linear, branched or cyclic C₁-C₁₂ alkyl group, wherein the alkyl group is optionally interrupted with one or more heteroatoms selected from the group consisting of O and N;
n is 1-10;
R⁴ is hydrogen or a linear, branched or cyclic C₁-C₁₂ alkyl group;
Y⁻ is a counter-anion, preferably a halogenide; and
R⁵ is a linear, branched or cyclic C₁-C₁₂ alkyl group, wherein the alkyl group is optionally interrupted with one or more heteroatoms selected from the group consisting of O and N;
and (b) an ethylenically unsaturated monomer, preferably an alkyl (meth)acrylate according to formula (II):

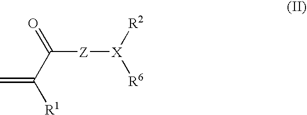

wherein:
R¹ is hydrogen or methyl;
X is O or N;
R² is hydrogen, a linear, branched or cyclic C₁-C₁₂ alkyl group or a substituent selected from the group defined for R⁶, provided that R² is absent when X═O;
R⁶ is hydrogen or a linear, branched or cyclic C₁-C ₁₂ alkyl group, wherein the alkyl group comprises at least an OH group or a —N(R¹)2 group; and
Z is either a direct bond or a linear, branched or cyclic C₁-C₁₂ alkyl group, wherein the alkyl group is optionally interrupted with one to three heteroatoms selected from the group consisting of O and N. Preferably, Z is a direct bond.

The present invention further relates to a process for making a hydrogel contact lens, wherein a monomer mixture comprising (a) a betaine ester of the formula (I) and (b) an alkyl (meth)acrylate according to formula (II) is polymerised.

The present invention also relates to the use of the betaine ester of the formula (I) for the manufacture of a hydrogel contact lens and as a wetting agent in a silicone hydrogel composition.

### DETAILED DESCRIPTION OF THE INVENTION

The verb “to comprise” as is used in this description and in the claims and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there is one and only one of the elements. The indefinite article “a” or “an” thus usually means “at least one”.

According to the invention, it is preferred that the monomer mixture further comprises (c) a carboxy betaine according to formula (III):

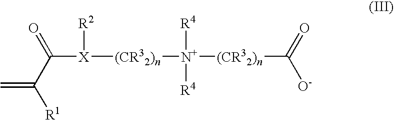

wherein:
R¹ is hydrogen or methyl;
X is O or N;
when X═N, R² is hydrogen or a linear, branched or cyclic C₁-C₁₂ alkyl group;
when X═O, R² is absent;
R³ is hydrogen, a linear, branched or cyclic C₁-C₁₂ alkyl group, wherein the alkyl group is optionally interrupted with one or more heteroatoms selected from the group consisting of O and N, preferably one to seven heteroatoms;
n is 1-10; and
R⁴ is hydrogen or a linear, branched or cyclic C₁-C₁₂ alkyl group.

The carboxy betaine according to formula (III) is shown as an inner salt structure. However, the carboxy betaine according to formula (III) may optionally contain an appropriate cation and an appropriate anion to render the carboxy betaine a neutral electrical charge as will be apparent to those skilled in the art.

According to a first preferred embodiment of the present invention, the monomer mixture comprises 1-50% by weight of (a) and 50-99% by weight of (b). However, according to a second preferred embodiment of the invention, the monomer mixture comprises 1-50% by weight of a blend of (a) and (c) and 50-99% by weight of (b), wherein the ratio of (a):(c) in the blend is between 1:99 and 99:1. As will be obvious to those skilled in the art, component (a), component (c) and the blend of (a) and (c) may optionally comprise other suitable monomers. Likewise, component (b) may comprise a single ethylenically unsaturated monomer, but also two or more.

The polymerisation reactions according to the present invention can be performed with radical initiators or precursors thereof, wherein the initiators or the precursors thereof are converted into radicals, either thermally or photochemically.

It is furthermore preferred that the monomer mixture comprises a cross-linking agent. Preferred cross-linking agents are selected from the group consisting of di(meth)acryloyl compounds, more preferably di(meth)acryloyl esters and di(meth)acryloyl amides. Even more preferably, the cross-linking agent is selected from the group consisting of di(meth)acrylate esters, wherein the group of di(meth)acrylate esters preferably consists of ethylene glycol di(meth)acrylate (EGDMA), diethyleneglycol di(meth)acrylate, allyl methacrylate (AMA), tetraethylene glycol di(meth)acrylate (TEGDA), polyethyleneoxide di(meth)acrylate (PEO-di(M)A), 1,3-propanediol di(meth)acrylate, 2,2-dimethyl-propanediol di(meth)acrylate, tripropylene glycol di(meth)acrylate, 1,3-butylene glycol di(meth)acrylate, sorbitol di(meth)acrylate and mixtures thereof.

Additionally, it is preferred that the monomer mixture comprises about 0.1% by weight to about 20.0% by weight, more preferably about 0.1% by weight to about 10.0% by weight, even more preferably about 0.1% by weight to about 8.0% by weight of the cross-linking agent, yet more preferably about 0.2% by weight to about 5.0% by weight, and most preferably about 0.4% by weight to about 3.0% by weight, based on the total weight of the monomer composition. In addition, it is preferred that the cross-linking agent comprises at least two cross-linking agents, preferably two cross-linking agents. The advantage of using at least two-cross-linking agents is that sufficient strength can be provided to the hydrogel contact lens if necessary. Furthermore, if two cross-linking agents are used, it is preferred that the molar ratio of these two cross-linking agents ranges from 1:99 to 99:1. It is also preferred that if two cross-linking agents are used, they are the cross-linking agents EGDMA and AMA.

The present invention also relates to a process for making a hydrogel contact lens, wherein a monomer mixture comprising (a) a betaine ester of the formula (I):

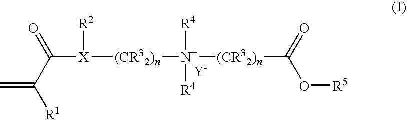

wherein:
R¹ is hydrogen or methyl;
X is O or N;
when X═N, R² is hydrogen or a linear, branched or cyclic C₁-C₁₂ alkyl group;
when X═O, R² is absent;
R³ is hydrogen, a linear, branched or cyclic C₁-C₁₂ alkyl group, wherein the alkyl group is optionally interrupted with one or more heteroatoms selected from the group consisting of O and N;
n is 1-10;
R⁴ is hydrogen or a linear, branched or cyclic C₁-C₁₂ alkyl group;
Y⁻ is a counter-anion, preferably a halogenide; and
R⁵ is a linear, branched or cyclic C₁-C₁₂ alkyl group, wherein the alkyl group is optionally interrupted with one or more heteroatoms selected from the group consisting of O and N;
and (b) an ethylenically unsaturated monomer, preferably an alkyl (meth)acrylate according to formula (II):

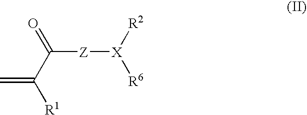

wherein:
R¹ is hydrogen or methyl;
X is O or N;
R² is hydrogen, a linear, branched or cyclic C₁-C₁₂ alkyl group or a substituent selected from the group defined for R⁶, provided that R² is absent when X═O;
R⁶ is hydrogen or a linear, branched or cyclic C₁-C₁₂ alkyl group, wherein the alkyl group comprises at least a OH group or a —N(R¹)2 group; and
Z is either a direct bond or a linear, branched or cyclic C₁-C₁₂ alkyl group, wherein the alkyl group is optionally interrupted with one to three heteroatoms selected from the group consisting of O and N; is polymerised.

Preferably, the monomer mixture employed in the polymerisation process further comprises (c) a carboxy betaine according to formula (III):

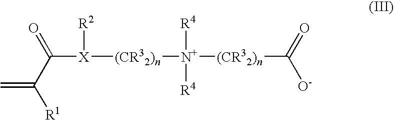

wherein:
R¹ is hydrogen or methyl;
X is O or N;
when X═N, R² is hydrogen or a linear, branched or cyclic C₁-C₁₂ alkyl group;
when X═O, R² is absent;
R³ is hydrogen, a linear, branched or cyclic C₁-C₁₂ alkyl group, wherein the alkyl group is optionally interrupted with one or more heteroatoms selected from the group consisting of O and N;
n is 1-10; and
R⁴ is hydrogen or a linear, branched or cyclic C₁-C₁₂ alkyl group.

A preferred group of the betaine esters according to formula (I) are those wherein X is N. This preferred group encompasses the more preferred group wherein R² is H.

A more preferred group of the betaine esters according to formula (I) are those wherein R² is hydrogen or a linear, branched or cyclic C₁-C₆ alkyl group. Even more preferably, R² is hydrogen or a methyl group and most preferably R² is a hydrogen atom.

Another preferred group of the betaine esters according to formula (I) are those wherein n is 1-6, more preferably 1-4 and most preferably n is 1-3.

Another preferred group of the betaine esters according to formula (I) are those wherein R³ is hydrogen or a linear, branched or cyclic C₁-C₆ alkyl group. Even more preferably, R³ is hydrogen or a methyl group and most preferably R³ is a hydrogen atom. Another preferred group for R³ is a —(C(R*)₂C(R*)₂Y)ₚ— group wherein p=1-12, R* is hydrogen or a linear C₁-C₆ alkyl group and Y is selected from the group consisting of O and N, wherein it is preferred that R* is H and/or that Y is O. Most preferably, R* is H and Y is O.

Yet another preferred group of the betaine esters according to formula (I) are those wherein R⁴ is hydrogen or a linear, branched or cyclic C₁-C₆ alkyl group. Even more preferably, R⁴ is hydrogen or a methyl group and most preferably R⁴ is a methyl group.

Additionally, another preferred group of the betaine esters according to formula (I) are those wherein R⁵ is a linear, branched or cyclic C₁-C₆ alkyl group, more preferably a C₁-C₃ alkyl group and most preferably R⁵ is a methyl group.

A yet another preferred group of the betaine esters according to formula (I) are those wherein R¹ is a methyl group.

According to the invention, a preferred group of the alkyl(meth)acylates according to formula (II) are those wherein X is O, which implies that the compounds according to formula (II) have no group R².

Another preferred group of the alkyl(meth)acylates according to formula (II) are those wherein R⁶ comprises an OH group. Preferably, R⁶ is a linear alkyl group, preferably an alkyl group comprising 1-6 carbon atoms. Even more preferably, R⁶ is a 2-hydroxy ethyl group (—CH₂—CH₂—OH). Most preferably, the compound according to formula (II) is hydroxyethyl methacrylate (HEMA).

According to the first preferred embodiment of the invention, it is preferred that the monomer mixture employed comprises 1-50% by weight of (a) and 50-99% by weight of (b). According to the second preferred embodiment of the invention, the monomer mixture comprises 1-50% by weight of a blend of (a) and (c) and 50-99% by weight of (b), wherein the ratio of (a):(c) in the blend is between 1:99 and 99:1.

The present invention also relates to the use of a betaine ester of the formula (I):

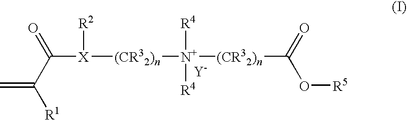

wherein:
R¹ is hydrogen or methyl;
X is O or N;
when X═N, R² is hydrogen or a linear, branched or cyclic C₁-C₁₂ alkyl group;
when X═O, R² is absent;
R³ is hydrogen, a linear, branched or cyclic C₁-C₁₂ alkyl group, wherein the alkyl group is optionally interrupted with one or more heteroatoms selected from the group consisting of O and N;
n is 1-10;
R⁴ is hydrogen or a linear, branched or cyclic C₁-C₁₂ alkyl group; and
Y⁻ is a counter-anion, preferably a halogenide; and
R⁵ is a linear, branched or cyclic C₁-C₁₂ alkyl group, wherein the alkyl group is optionally interrupted with one or more heteroatoms selected from the group consisting of O and N;
for the manufacture of a hydrogel contact lens.

The present invention furthermore relates to the use of a betaine ester of the formula (I) as a wetting agent in a silicone hydrogel composition, in particular an internal wetting agent.

Preferably, the water content of the hydrogel contact lens is about 30% by weight to about 80% by weight, based on the weight of the hydrogel contact lens, and preferably in the range of about 35% by weight to about 75% by weight.

### Examples

### Example 1

### Synthesis of the carboxy betaine ester N-Carbomethoxymethyl-N-methacryloylamidopropyl-N,N-dimethylammoniumchloride

The synthesis of this carboxy betaine ester, denoted AMIM, is based on the disclosure of U.S. Pat. No. 2,777,872 of E.I. DuPont de Nemours.

### Synthesis Route:

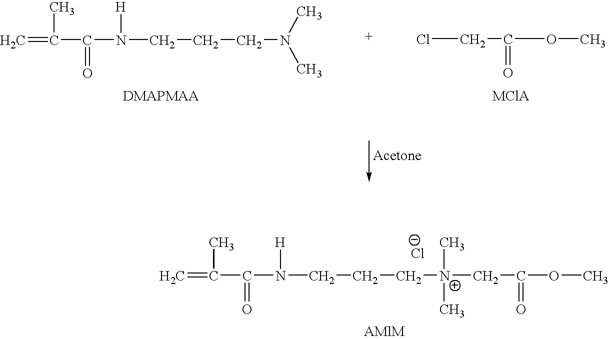

The synthesis is performed under a dry nitrogen atmosphere in dry acetone. Under these circumstances the carboxy betaine ester, which is a salt, precipitates from the solution. Being salts, the carboxy betaine esters are very hygroscopic since they absorb water rather quickly and thus must be stored under dry conditions to retain their quality.

Synthesis: 42.566 g (0.250 mol) N-[3-(dimethylamino)propyl]-methacrylamide (DMAPMAA) in 100 ml of dry acetone was added to a three necked flask and stirred. Then, 29.848 g (0.275 mol) methylchloro acetate (MClA) in 30 ml dry acetone was added to a dropping funnel attached to the three necked flask. The reaction was commenced under a continuous slight dry nitrogen overpressure while the tip of the reaction flask touched the ice/water mixture, thus setting the initial temperature of the reaction solution at 15° C. Now, MClA was slowly added in about 30 minutes, while maintaining the reaction temperature just below 15° C. by varying the immersion depth of the reaction flask in the ice/water mixture. During the addition of MClA only a slight whitish haze developed in the reaction solution. After addition of all MClA, the reaction flask was lifted from the ice/water bath, and the reaction solution was allowed to warm to ambient temperature while still being stirred. Then the temperature slowly rose to about 30° C., slightly over ambient temperature, while more and more white crystals were formed. The temperature was controlled to avoid an increase above 30° C. in order to prevent that too much acetone evaporated from the reaction solution. After one night of continuous stirring, a dense crust of white crystalline material had precipitated on the inside of the reaction flask, while a slurry of crystals had formed in the reaction solution.

The crystals were isolated by filtration and washed with dry acetone under a flow of dry nitrogen gas to avoid water uptake. The crystals were dried in a vacuum oven at 50° C. for one night. The yield was 60.215 g (79%). The identity was confirmed by means of ¹H-NMR (300 MHz) in D₂O and FT-IR. Its purity was estimated as 99+%.

### Example 2

Formulation for moulding a Contact Lens. A formulation was developed yielding a Contact Lens containing a water content of about 55wt % with the desired mechanical and optical lens properties. For a 200 g formulation batch, 31.5 g (14.96.% by weight) AMIM, 175.0 g (83.10% by weight) HEMA (hydroxyethyl methacrylate), 0.5 g (0.24% by weight) EGDMA (ethylene glycol dimethacrylate), 0.8 g (0.38% by weight) AMA (allyl methacrylate), 2.5 (1.18 wt %) UV-Blocker (a benzotriazole based UV-blocker like 2-(2′-Hydroxy-5′-methacryloxy-ethylphenyl)-2H-benzotriazol) and 70 ppm of a lens handling tint material were combined and stirred until all solids had dissolved. Then the solution was filtered over a PI glass filter into a clean storage jar and stored at about 5° C. until further use. Just before use of a previously prepared formulation as described above, about 0.320 g (typically about 0.200% by weight (completing the total content of substances in the formulation to 100wt %) of a thermal radical initiator like V65 (WAKO) was added and the formulation was stirred until all solids had dissolved.

For moulding, the solution was dispensed into a lower polypropylene mould half, then an upper polypropylene mould half was placed on top of the lower mould half. These halves enclose a cavity in the form of a contact lens. The solution in the mould cavity was cured for about half an hour at about 95° C., after which the mould was opened, the lens was removed and hydrated in a saline solution. After autoclaving the lens in this same hydrating saline solution, the lenses were inspected for quality and found to be within specification.

1. A hydrogel contact lens comprising a polymer composition obtainable by polymerising a monomer mixture comprising:
(a) a betaine ester of the formula (I): 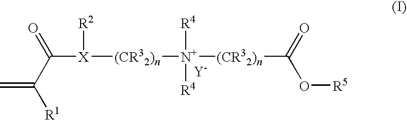
wherein:
R¹ is hydrogen or methyl;
X is O or N;
when X═N, R² is hydrogen or a linear, branched or cyclic C₁-C₁₂ alkyl group;
and when X═O, R² is absent;
R³ is hydrogen, a linear, branched or cyclic C₁-C₁₂ alkyl group, wherein the alkyl group is optionally interrupted with one or more heteroatoms selected from the group consisting of O and N;
n is 1-10;
R⁴ is hydrogen or a linear, branched or cyclic C₁-C₁₂ alkyl group;
Y⁻ is a counter-anion; and
R⁵ is a linear, branched or cyclic C₁-C₁₂ alkyl group, wherein the alkyl group is optionally interrupted with one or more heteroatoms selected from the group consisting of O and N; and
(b) an ethylenically unsaturated monomer.

2. The hydrogel contact lens according to claim 1, wherein the ethylenically unsaturated monomer is an alkyl(meth)acrylate according to formula (II): 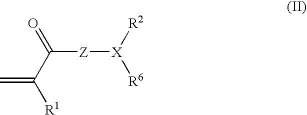
wherein:
R¹ is hydrogen or methyl;
X is O or N;
R² is hydrogen, a linear, branched or cyclic C₁-C₁₂ alkyl group or a substituent selected from the group defined for R⁶, provided that R² is absent when X═O;
R⁶ is hydrogen or a linear, branched or cyclic C₁-C₁₂ alkyl group, wherein the alkyl group comprises at least an —OH group or an —N(R¹)2 group; and
Z is either a direct bond or a linear, branched or cyclic C₁-C₁₂ alkyl group, wherein the alkyl group is optionally interrupted with one to three heteroatoms selected from the group consisting of O and N.

3. A hydrogel contact lens according to claim 1, wherein the monomer mixture further comprises (c) a carboxy betaine according to formula (III): 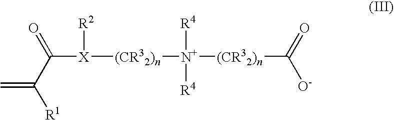
wherein:
R¹ is hydrogen or methyl;
X is O or N;
when X═N, R² is hydrogen or a linear, branched or cyclic C₁-C₁₂ alkyl group;
and when X═O, R² is absent;
R³ is hydrogen, a linear, branched or cyclic C₁-C₁₂ alkyl group, wherein the alkyl group is optionally interrupted with one or more heteroatoms selected from the group consisting of O and N;
n is 1-10; and
R⁴ is hydrogen or a linear, branched or cyclic C₁-C₁₂ alkyl group.

4. The hydrogel contact lens according to claim 1, wherein the monomer mixture comprises 1-50% by weight of monomer (a) and 50-99% by weight of monomer (b).

5. The hydrogel contact lens according to claim 1, wherein the monomer mixture comprises 1-50% by weight of a blend of monomers (a) and (c); and 50-99% by weight of monomer (b), wherein the ratio of (a):(c) in the blend is between 1:99 and 99:1.

6. The process for the manufacture of a hydrogel contact lens, the process comprising polymerizing a monomer mixture comprising:
(a) a betaine ester of the formula (I): 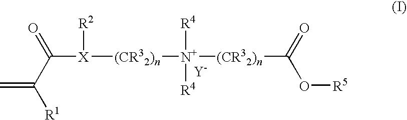
wherein:
R¹ is hydrogen or methyl;
X is O or N;
when X═N, R² is hydrogen or a linear, branched or cyclic C₁-C₁₂ alkyl group;
and when X═O, R² is absent;
R³ is hydrogen, a linear, branched or cyclic C₁-C₁₂ alkyl group, wherein the alkyl group is optionally interrupted with one or more heteroatoms selected from the group consisting of O and N;
n is 1-10;
R⁴ is hydrogen or a linear, branched or cyclic C₁-C₁₂ alkyl group;
Y⁻ is a counter-anion; and
R⁵ is a linear, branched or cyclic C₁-C₁₂ alkyl group, wherein the alkyl group is optionally interrupted with one or more heteroatoms selected from the group consisting of O and N; and
(b) an ethylenically unsaturated monomer.

7. The process according to claim 6, wherein the ethylenically unsaturated monomer is an alkyl(meth)acrylate according to formula (II): 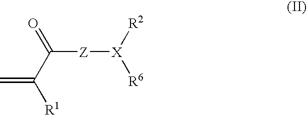
wherein:
R¹ is hydrogen or methyl;
X is O or N;
R² is hydrogen, a linear, branched or cyclic C₁-C₁₂ alkyl group or a substituent selected from the group defined for R⁶, provided that R² is absent when X═O; and
R⁶ is hydrogen or a linear, branched or cyclic C₁-C₁₂ alkyl group, wherein the alkyl group comprises at least an —OH group or an —N(R¹)2 group.

8. The process according to claim 6, wherein the monomer mixture further comprises (c) a carboxy betaine according to formula (III): 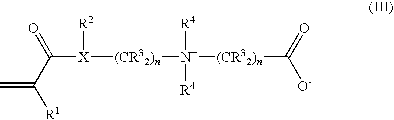
wherein:
R¹ is hydrogen or methyl;
X is O or N;
when X═N, R² is hydrogen or a linear, branched or cyclic C₁-C₁₂ alkyl group;
and when X═O, R² is absent;
R³ is hydrogen, a linear, branched or cyclic C₁-C₁₂ alkyl group, wherein the alkyl group is optionally interrupted with one or more heteroatoms selected from the group consisting of O and N;
n is 1-10; and
R⁴ is hydrogen or a linear, branched or cyclic C₁-C₁₂ alkyl group.

9. The process according to claim 6, wherein the monomer mixture comprises 1-50% by weight of monomer (a) and 50-99% by weight of monomer (b).

10. The process according to claim 6, wherein the monomer mixture comprises 1-50% by weight of a blend of monomers (a) and (c) and 50-99% by weight of monomer (b), wherein the ratio of (a):(c) in the blend is between 1:99 and 99:1.

11. A hydrogel contact lens comprising a betaine ester of the formula (I): 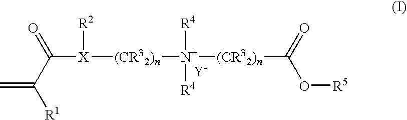
wherein:
R¹ is hydrogen or methyl;
X is O or N;
when X═N, R² is hydrogen or a linear, branched or cyclic C₁-C₁₂ alkyl group;
and when X═O, R² is absent;
R³ is hydrogen, a linear, branched or cyclic C₁-C₁₂ alkyl group, wherein the alkyl group is optionally interrupted with one or more heteroatoms selected from the group consisting of O and N;
n is 1-10;
R⁴ is hydrogen or a linear, branched or cyclic C₁-C₁₂ alkyl group; and
Y⁻ is a counter-anion; and
R⁵ is a linear, branched or cyclic C₁-C₁₂ alkyl group, wherein the alkyl group is optionally interrupted with one or more heteroatoms selected from the group consisting of O and N.

12. A method of wetting a silicone hydrogel composition, the method comprising incorporating with the silicone hydrogel composition a wetting agent comprising a betaine ester of the formula (I): 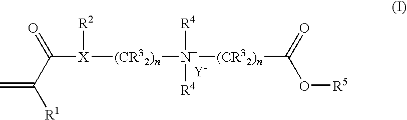
wherein:
R¹ is hydrogen or methyl;
X is O or N;
when X═N, R² is hydrogen or a linear, branched or cyclic C₁-C₁₂ alkyl group;
when X═O, R² is absent;
R³ is hydrogen, a linear, branched or cyclic C₁-C₁₂ alkyl group, wherein the alkyl group is optionally interrupted with one or more heteroatoms selected from the group consisting of O and N;
n is 1-10;
R⁴ is hydrogen or a linear, branched or cyclic C₁-C₁₂ alkyl group; and
Y⁻ is a counter-anion; and
R⁵ is a linear, branched or cyclic C₁-C₁₂ alkyl group, wherein the alkyl group is optionally interrupted with one or more heteroatoms selected from the group consisting of O and N;.

13. The method according to claim 12, wherein the wetting agent is an internal wetting agent.
